(19) United States
(12) Patent Application Publication
(10) Pub. No.: US 2020/0146848 A1
(43) Pub. Date: May 14, 2020

(54) METHOD FOR CAPTURING SENSOR DATA

(71) Applicant: Ottobock SE & Co. KGaA, Duderstadt (DE)

(72) Inventors: Filip SZUFNAROWSKI, Gdynia (PL); Erik ALBRECHT-LAATSCH, Rosdorf (DE); Michael NOLTE, Seeburg (DE); Christian WILL, Göttingen (DE); Jens DRIESEBERG, Duenwald (DE); Christopher BARTELS-SELLERING, Göttingen (DE)

(73) Assignee: Ottobock SE & Co. KGaA, Duderstadt (DE)

(21) Appl. No.: 16/614,019

(22) PCT Filed: Mar. 16, 2018
(86) PCT No.: PCT/EP2018/056754

§ 371 Date: Nov. 15, 2019

(30) Foreign Application Priority Data
May 17, 2017 (DE) 10 2017 110 761.4

(51) Int. Cl. A61F 2/68; A61F 2/60; A61F 5/01; A61B 5/11; A61B 5/00

(52) U.S. Cl. CPC A61F 2/68; A61F 2/60; A61F 5/0123; A61F 2002/7625; A61B 5/4851; A61F 2002/689; A61B 5/112

(57) ABSTRACT

A method includes fixing at least one sensor to a body part of a person, sending signals, with the at least one sensor, to an electronic data processing device, the signals containing measurement data and an individual sensor identifier, and determining, with the electronic data processing device, the body part on which the sensor is arranged or is to be arranged, and allocating the measurement data of the at least one sensor to the body part.

The invention relates to a method in which at least one sensor is fixed to a body part of a person. It also relates to a system for conducting such a method.

The set-up of a prosthesis, especially a leg prosthesis, must be executed with care and in particular, it must be individually adjusted to the respective patient. This applies to both upper leg amputees (transfemoral) and lower leg amputees (transtibial) who still have a natural knee joint on the leg that is to be treated. Of course, this also applies to all other prostheses. The set-up of the prosthesis must be conducted in such a way that, for instance, as natural a gait pattern as possible is achieved, so the wearer of the prosthesis need not make any uncomfortable, unusual or unnatural movements to control the prosthesis. At the same time, it must be ensured that the prosthesis provides its wearer with as high a degree of safety as possible in as many situations as possible.

Conventionally, a static set-up is conducted on a standing patient and a dynamic set-up while the patient is walking. With regards to a static set-up, there are systems and platforms available to the orthopedic technician who also conducts the set-up which provide objective measurement data; however, a dynamic set-up is still often conducted on the basis of highly subjective data that the competent orthopedic technician establishes himself, for example upon observing the gait pattern. This is particularly impractical if, for example due to spatial restrictions, the orthopedic technician cannot adopt the necessary perspectives and cannot, for instance, observe the patient from the side while the patient is walking.

To improve the quality of a dynamic prosthetic set-up, it would be beneficial to be able to provide a system which would enable the fixing of different sensors, which can record the required measured values, at different points on the patient's body and/or at different positions of the prosthesis. The corresponding sensors could measure, for example, inertial data such as absolute angles or relative angles, for instance the angle of a knee, accelerations or speed, which provide objective information on the gait pattern and the current set-up of the prosthesis. However, in order to generate reliable data, it is necessary to know—as precisely as possible—the orientation of the sensors on the body or the prosthesis and to ensure that this orientation and/or configuration changes as little as possible during walking and dynamic analysis; preferably, said orientation and/or configuration does not change at all.

DE 10 2008 024 746 A1 describes an orthopedic device for lower limbs, wherein precisely this orientation and alignment of a sensor on the prosthesis should be determined. To this end, the system must first be brought into a learning mode, in which certain data can be recorded and evaluated in such a way that any existing errors and inaccuracies in the alignment and/or positioning of the sensor can be recognized and factored out of the of the calculated measurement data. It is only once this has been done that it can be switched to operating mode, in which the sensors identify the values required.

However, it is not possible to check of the orientation and/or alignment of the sensors in this manner if the learning mode is not reactivated at different times. This is complex and not done in practice.

Therefore, the invention aims to propose a method by means of which a dynamic prosthesis set-up can be conducted simply, reliably and objectively.

The invention solves the problem by way of a method in which at least one sensor is fixed to a body part of a person; the at least one sensor sends signals, which contain the measurement data and an individual sensor identifier, to an electronic data processing device; and the electronic data processing device determines the body part on which the sensor is arranged or is to be arranged and allocates the measurement data of the at least one sensor to the body part. This allocation preferably occurs automatically. To this end, the electronic data processing device preferably recognizes the individual sensor identifier.

Here, a body part should be understood to mean both a natural body part of a person and a component of an orthosis or prosthesis worn by the person.

By way of the method, the signals emitted by the sensor, said signals containing the measurement data in particular, are allocated to the correct body part on which the sensor is situated. This renders possible an objective logging of measurement data, which allows an objective dynamic set-up of a prosthesis and thus results in an improved quality of the various prostheses. The at least one sensor can be allocated to different body parts, which can be recognized or detected by the electronic data processing device, such that the respective sensor can be used on different body parts for different patients. In particular, in the event that several sensors are allocated to different body parts, it is no longer necessary, on the one hand, to provide a large number of sensors for the individual body parts and, on the other hand, to employ a complicated and elaborate method which would render it always necessary to fix a corresponding sensor to the specific body part. On the one hand, this is complex and impractical for the orthopedic technician and, on the other hand, prone to error. The method according to the invention prevents these disadvantages.

In a preferred embodiment, the at least one sensor does not send the individual sensor identification until it is instructed to do so. This instruction is preferably given via a corresponding control signal that is emitted by the electronic data processing device and detected by the at least one sensor. If several sensors are used, the control signal preferably contains information on the sensor that is being spoken to and that should communicate its individual sensor identifier.

Alternatively, the at least one sensor can also feature an activation device, such as a button. If the activation device is activated, the sensor sends its individual sensor identifier, which is detected by the electronic data processing device.

In a preferred embodiment of the method, at least one detector is allocated to the electronic data processing device, said detector detecting at least one part of the signals from the sensor, wherein the electronic data processing device determines the body part using the detected signals, especially a direction or position from which the individual identifier is sent, or from a time curve of the measurement data.

The sensor identifier can be transmitted via visible light, for example. It is practical if this achieved via at least one LED, preferably several LEDs, which especially preferably emit light in different colors. The individual sensor identifier may comprise combinations of flashing frequency, colors, intensity and/or patterns, a certain sequence or a code made up of one or several of these parameters. If the individual sensor identifier is emitted by a sensor, it can be captured by a corresponding detector, which is allocated to the data processing device and, in the example of an embodiment given, may be a camera. Of course, other light-sensitive sensors are conceivable. Alternatively or additionally, the sensor identifier may also contain non-visible electromagnetic radiation, for example in the infra-red range, and/or audible and/or inaudible acoustic signals, for instance in the ultrasound range. Correspondingly, the detector must be able to detect at least a part of this individual sensor identifier, wherein said part enables the electronic data processing device to identify the sensor with the aid of the detected parts of the individual sensor identifier. Preferably, the at least one detector is able to detect the complete sensor identifier.

However, the at least one detector, which is allocated to the data processing device, is preferably able to detect not only the individual sensor identifier, but also additional information from which the position of the sending sensor can be determined. For instance, this may be the direction from which the sensor identifier is sent, so that the electronic data processing device is able to determine the body part on which the sensor is arranged. Preferably, the electronic data processing device subsequently allocates the individual sensor identifier of the sending sensor to the respective body part, such that, when the sensor is in use, the transmitted measurement data can be identified as coming from this sensor and allocated to the respective body part.

It is beneficial if the individual sensor identifier is transmitted to the respective sensor before said sensor is fixed to the body part. Alternatively, the identifier can also be transmitted to the sensor if it has already been fixed to a body part of the person. The at least one sensor preferably receives its individual identifier from a transmission device, said transmission device being connected to the electronic data processing device for this purpose, and can preferably transmit a verification via a suitable communication protocol of the electronic data processing device. Using this verification, which preferably contains the individual sensor identifier, or using the otherwise transmitted signals, the electronic data processing device can preferably deduce the position and/or the direction that the sensor is currently in, and thus also determine the body part of the person.

Alternatively or additionally to this procedure, the electronic data processing device may use the detected signals, such as the time curve of the measurement data, to determine the body part on which the sensor is situated. Here, it is practical if the electronic data processing device contains information concerning the movement sequence, for example walking on level ground, walking on a slanted surface, climbing steps, sitting down, running or standing up, to which the detected time curves of the measurement data belong. By comparing stored time curves of measurement data, the electronic data processing device is able to recognize on which body part the sensor is located, said sensor being the one to which this detected measurement data belongs.

Alternatively or additionally, other classification algorithms may be used to compare stored time curves of measurement data. For instance, this may comprise a simple sorting according to threshold values or a complex neural network that is able to make autonomous decisions.

The sensor signals also preferably contain information on the body part on which the sensor is located. Said signals can be set or identified in a range of different ways. In an especially preferred configuration, the sensor or a housing, in which the sensor is arranged, features an adjustment device, such as a control dial or a slide controller. By adjusting this adjustment device, the information concerning the body part contained in the sensor signals is encoded. The individual adjustment options, such as a slide controller or control dial, are assigned to the various body parts of the person, such that the person who fixed or will fix the sensor to the body part also adjusts the adjustment device accordingly. Alternatively or additionally, the sensor may be fixed to the body part by means of a bracket, which has an effect on the signals sent by the sensor. The bracket is preferably adapted to the respective body part of the person. Of course, a bracket for a sensor that is to be arranged, for instance, on the torso of a person is configured differently to a bracket for a sensor that is to be arranged on the upper arm, the wrist or the knee. Since different brackets must be used for the different body parts, the signal concerning the body part emitted by the sensor can be adjusted in the bracket, for instance via an electrical circuit or an RFID chip. Where applicable, it is also beneficial to provide a switch or an adjustment device on the bracket in this case, wherein said switch or adjustment device can be used to encode whether the bracket is arranged on a left or a right-hand body part.

In a particularly simple configuration, the sensor can take the information on the body part from the respective bracket.

The bracket preferably detects the body part to which it is fixed. This may occur, for example, by measuring the circumference of the body part. For instance, if the bracket has an extractable or length-adjustable belt, the length of the belt that is required to encircle and/or enclose the body part can be used to determine what type of body part this relates to. In this case, if there is more than one of the pertinent body part, it is only necessary to establish, for example, whether the body part is the right or left one, for instance of a leg or an arm. Alternatively, printed barcodes or other codes, position markers, glyphs or other such elements can be used.

The bracket is preferably configured to recognize the type of sensor that is fixed to it. The bracket is thus able to transmit information on body parts and/or sensors, wherein such information can be sent either by the bracket itself to the electronic data processing device or via the sensor.

The sensor preferably has an adjustment device, by way of which the information on the body part can be adjusted. This may refer, for example, to a manually activated element, such as a control dial or a slide controller. Depending on the body part on which the sensor is arranged, these adjustment elements are brought into a certain position, by way of which the information on the body part can be adjusted, said information containing this position of the sensor.

The at least one sensor preferably only sends the signals to the electronic data processing device if it has received a corresponding request signal. This renders it possible to ensure that the electronic data processing device only receives signals from a single sensor and not several signals of different sensors that are fixed to different body parts of the person, overlap each other and, potentially, cannot be evaluated.

It is preferable if at least two sensors are fixed to different body parts.

In a preferred embodiment, these at least two sensors are configured to detect information on distances between two of these sensors and to send said information in the signals sent to the electronic data processing device. In this case, the electronic data processing device can use stored distance patterns to evaluate the individual positions and body parts to which the sensors are fixed.

When determining information on distances between two sensors, it is beneficial if at least one sensor is, preferably all the sensors are, able to emit corresponding test signals, for example in the form of vibrations, electromagnetic radiation or sound, wherein said signals can be detected by the respective other sensors. It has proven to be practical if the sensors which receive corresponding test signals emit a corresponding response signal. Run-times of the test signal and corresponding response signal can be used to determine the distances of the various sensors among one another and thus determine an arrangement of the sensors relative to one another. Since the possible body parts to which the sensors can be fixed are known, this information can be used to determine which sensor is arranged on which body part. Furthermore, if the test signal from the original sending sensor is sent specifically in a certain direction or in a certain spatial region, a distinction can also be made between right and left-hand body parts, such as legs or arms.

Alternatively or additionally, the sensors can emit test signals that are detected by other sensors. Information on the signals detected in this way, especially the times of detection, can then either be transmitted from the detecting sensors to the electronic data processing device or transmitted to the emitting sensor in the form of response signals. It is preferable if, as an alternative or in addition to this, they are received by the electronic data processing device.

It is beneficial for the sensors to be able to determine a distance from an object, such as the ground. This may occur, for instance, via ultrasound signals that are emitted towards the ground. The reflected ultrasound signals can be detected and the distance from the ground determined by way of the run-time that is determined using said reflected signals and the known speed. This renders it possible to establish, for instance, whether a sensor is arranged on a knee, an upper leg or a shoulder. Of course, other body parts can be identified, provided that they are at different distances from the ground. Alternatively or additionally, a positioning system that operates in a precise manner, such as a Global Positioning System (GPS), can be used to detect—as accurately as possible—the position of the individual sensors and thereby establish the body part on which the sensors are arranged.

Physical signals coming from the person may also be used to determine positions of the individual sensors relative to one another or relative to a certain point on the body of the wearer. For example, the pulse rate, heart rate, corresponding blood pressure and/or the ECG can be used to determine, for instance, a distance of the sensor from the person's heart. Of course, all other biosignals that are emitted by the body and that allow a location and/or distance to be determined, may also be used on their own or in combination with other signals described here.

The invention also solves the problem by way of a method in which at least one sensor is fixed to a body part of a person; the at least one sensor sends signals, which contain the measurement data, to an electronic data processing device; the electronic data processing device emits a control signal to the at least one sensor; and the at least one sensor, in response to the control signal, emits a response signal that can be detected by a person. This response signal, such as an audio signal, a haptic signal and/or a visual signal, is detected by the person, for example an orthopedic technician, who then preferably communicates the position of the corresponding sensor to the electronic data processing device. To this end, said data processing device has an interface via which a person can enter relevant data into the data processing device.

The invention also solves the problem by way of a system with at least one sensor and one electronic data processing device that is configured to carry out such a method.

Of course, the at least one sensor must be fixed to a prosthesis or an orthosis. These elements then preferably form a system with an orthosis and/or a prosthesis and a bracket for fixing a sensor to an object, wherein the bracket features at least two fixing elements for fixing the bracket on the object, said fixing elements being separated from one another by at least one spacer element, and at least one sensor fixing element for fixing the sensor to the bracket, wherein the at least one spacer element exhibits a smaller bending stiffness in a first direction than in a second direction, which is perpendicular to the first direction.

Such a system is a separate invention in and of itself or may be used in combination with other properties described here. This also applies for a bracket for such a system.

In general, sensor are fixed to a body part or part of a prosthesis via a belt, for example, which is placed around the respective body part or part of the prosthesis. Often, this may cause the sensor to twist relative to the body part, such as an upper leg, wherein this twisting should be recognized and quantified to enable an optimal evaluation of the measurement data identified by the sensor. Furthermore, vibrations and accelerations which occur while walking may cause displacements or slipping of the sensors relative to the body part or the part of the prosthesis.

Here, a body part should be understood to mean both a natural body part of a person and a component of an orthosis or prosthesis worn by the person.

The two fixing elements provided according to the invention, which may be provided in the form of two belts, for instance, that are placed around the respective body part or part of the prosthesis, allow for the definition of the orientation of the spacer element provided between the two fixing elements. A rotation or displacement of this element relative to the body part or part of the prosthesis, for example relative to the upper leg, is only possible to a very limited extent; it is preferably not possible at all. As a result, the orientation of at least the spacer element relative to the object, i.e. especially relative to the body part or part of the prosthesis, is defined and cannot be changed, or can only be done so to a marginal extent. The sensor itself is fixed to the bracket, especially preferably to the spacer element, via the sensor fixing element. By way of markers provided, for instance, on the sensor or sensor housing in which the sensor is situated, a relative orientation of the sensor housing or the sensor relative to the spacer element can be easily read and, preferably, adjusted. This renders it possible to determine and define the fixing of the sensor to the object, i.e. preferably to the body part or the part of the prosthesis, the alignment and the orientation of the sensor relative to this object.

Due to the special configuration of the spacer element with the anisotropic bending stiffness, the retention of an optimal orientation of the sensor can be ensured whilst still enabling a movement in another direction. A sensor may be, for example, an acceleration sensor or an angle sensor that should be aligned with an already known fixed longitudinal axis on the sensor, for example parallel to the object, for instance an upper leg, in order to obtain optimal measurement data. This is easily achieved with a bracket according to the invention. The two fixing elements are fixed at two positions on the upper leg, wherein said positions are separated from one another by the spacer element. This ensures that the spacer element extends parallel to the upper leg, for example to the femur. The sensor, which is preferably already fixed to the bracket or can be fixed to it later, thus has an already known, preferably adjustable, alignment relative to the spacer element and therefore relative to the upper leg. The imaginary longitudinal axis of the upper leg and the corresponding direction of the sensor that is to be determined run parallel and therefore span one plane. The spacer element now exhibits different degrees of bending stiffness in two different directions, which are preferably configured in such a way that the bending stiffness is particularly high for bends of the spacer element that move the pre-defined sensor direction out of the plane it spans along with the symmetrical axis of the upper leg, such that this type of movement, which would alter the position and thus reduce the quality of the measurement data, is difficult or preferably not possible at all. Conversely, it is considerably easier in a perpendicular direction, which preferably does not guide the pre-defined direction of the sensor out of the plane it spans along with the symmetrical axis of the upper leg, since here the bending stiffness is reduced.

This is of course not limited to examples of an embodiment which feature a sensor fixed to an upper leg of a person.

The at least one spacer element is preferably a compressive force transmission element. This ensures that the two fixing elements are always at the same distance from one another and that the spacer element is rigid.

In a preferred embodiment, the at least one spacer element has at least two individual elements that run parallel alongside one another, said elements being bars, braces, rods or tubes in particular. This renders it especially easy to achieve the various degree of bending stiffness, as the individual elements themselves may comprise an isotropic bending stiffness, i.e. one that is the same in all directions, and the desired various degrees of bending stiffness of the spacer element are only achieved via the positioning of the various individual elements alongside one another.

It has proven to be especially beneficial if the individual elements are designed to be integral with one another. It is especially preferable if the entire spacer element, especially preferably the entire bracket, is designed as a single piece. Said bracket may be, for instance, a plastic element which has been, for example, cut out, sawn out or punched out of a plastic panel. Alternatively, 3D-print components or metal components may also be used. In a preferred embodiment, the individual elements are made from a plastic or a fiber composite, in particular a carbon fiber composite or a glass fiber composite. This is particularly practical if the individual elements and the spacer elements are not designed as a single piece. In this case, carbon fiber rods or glass fiber rods may be used, said rods being easy to produce, having a high degree of bending stiffness and yet still having a low net weight.

The at least one sensor fixing element is preferably arranged on the at least one spacer element. Here, it has proven particularly preferable if the sensor fixing element is arranged in the middle between the two fixing elements.

It is beneficial if the sensor fixing element is arranged, preferably clamped or clipped, on the at least one spacer element such that it can be detached. As a result, the sensor used can be very easily exchanged if this is necessary due to the measurement data required or for maintenance and/or repair of the sensor. Furthermore, the sensor fixing element can be arranged along the longitudinal distance of the spacer element such that it can be displaced, so that the position of the sensor on the object, i.e. for example on the body part or the part of the prosthesis, can also be adjusted later and brought into the optimal position. At the same time, this does not have an effect on the orientation of the at least one sensor.

The object preferably has a longitudinal direction and is, in particular, a prosthetic component, an orthosis component or a part of a human body. The bracket is preferably designed in such a way that a direction in which the at least two fixing elements are situated at a distance from one another corresponds to the longitudinal direction or runs parallel to it when the bracket is arranged on the object. This direction preferably corresponds to a direction of extension of the spacer element. If the bracket is arranged, for instance, on a prosthetic component or a part of a human body, this ensures that the orientation of the bracket and therefore preferably also the orientation of the sensor relative to the object is fixed, so that an incorrect operation and resulting imprecise or incorrect measured values are prevented or at least reduced.

The at least one sensor preferably comprises an inertial sensor, a spatial position sensor—in particular an electromagnetic one—an angle sensor, an acceleration sensor, a sensor for detecting bio-signals of the human body, such as ECG, blood pressure, pulse etc., and/or an electromagnetic tracker.

A system comprising such a bracket and a sensor that is fixed to the at least one sensor fixing element also constitutes an independent invention, which can be used with other properties described here. Preferably, the at least one sensor fixing element and the at least one sensor are designed in such a way that the sensor can only be fixed on the sensor fixing element in a few, preferably two, especially preferably only one orientation. This ensures that the sensor can only be fixed in certain orientations, such that the orientation of the sensor relative to the bracket and therefore relative to the spacer element or to the sensor fixing element need not be detected, checked and, where applicable, adjusted and also so that it does not constitute a source of measurement errors or other such inaccuracies.

If the prosthesis set-up is to be examined dynamically, it is practical to be able to arrange sensors at different points and on different body parts of the person. An electronic data processing device is then preferably able to allocate measurement data and signals, which in particular contain an individual sensor identifier, to the body part to which the respective sensor is fixed.

The set-up and/or configuration of an orthosis and/or prosthesis is essential with regards to the level of comfort and functionality. It is therefore beneficial to check the device. This is achieved by way of a method for fitting a prosthesis or orthosis or for determining malpositions in the set-up of a prosthesis or orthosis or the lower limb comprising the steps a) recording of a first set of measurement data of at least one sensor that is fixed to a body part of a person, wherein the first set of measurement data is allocated to a first state of movement of the person, b) recording of a second set of measurement data of at least one sensor that is fixed to a body part of the person, wherein the second set of data is allocated to a second state of movement of the person and c) evaluation of the first and second set of measurement data.

This method is a separate invention in and of itself or may be used in combination with other properties described here.

This method is based on the knowledge that, to ensure an optimal prosthesis set-up, it is often necessary to record measurement data from sensors in different states of movement and to simultaneously evaluate said data. The first state of movement and the second state of movement thus differ from one another. As a result, information is obtained that cannot be obtained via a simple evaluation of the measurement data of just one of the two states of movement. Here, it is of course necessary for the two states of movement to differ from one another.

The method can be conducted entirely by a computer or an electronic data processing device, provided that said device has access to the first and second set of recorded measurement data. This measurement data is transmitted by the respective at least one sensor of the electronic data processing device and preferably stored in an electronic memory.

In a preferred embodiment, the second state of movement is selected on the basis of the first set of measurement data. Preferably, the first set of measurement data is evaluated first. Here, indications of present malpositions may be detected already, wherein said malpositions can be verified, confirmed or disproved by way of measurements in a second state of movement. It is therefore often beneficial to first of all select the first set of measurement data so as to select the optimal second state of movement, such that any remaining uncertainties and/or ambiguities from the first set of measurement data can be eliminated using the second set of measurement data.

Alternatively, it is of course also possible for a fixed second state of movement to be assigned to a selected first state of movement. If the first state of movement is climbing stairs, for example, it may be practical to assign to it a movement downstairs or walking on a slanted plane, i.e. an incline or a decline. This type of specific assignment renders it possible to determine a multitude of different malpositions from the measurement data. A specific assignment is practical if a single second state of movement can be assigned to a first state of movement. If, in this case, there are any ambiguities and different evaluation results from the evaluation of the first set of measurement data may allow for different second states of movement, such a specific assignment is no longer practical.

For certain prostheses and/or patients, it is also practical to use more than two states of movement and to record separate measurement data in each of these numerous states of movement. In this case, it is beneficial to select a fixed sequence of the different states of movement and to allocate the relevant recorded measurement data to the corresponding current state of movement.

Preferably, the selected second state of movement is displayed via a communication device, especially by way of an audio signal and/or a haptic signal and/or a visual signal. A device by means of which the method can be conducted thus has a communication device, such as a display or a display device, which may be in the form of different colored LEDs, lights or other display elements, for example. Of course, a speaker may be provided that can be used to emit audio signals, such as spoken words. The communication device preferably also features a microphone that enables a voice input in the form of commands or instructions. Once the first set of measurement data has been evaluated and the second state of movement has been detected on the basis of this evaluation, the second state of movement is communicated to a user of the device, such as an orthopedic technician via the communication device. The patent is then instructed, for example by way of the communication device or by an orthopedic technician, to assume the next state of movement and, for instance, to climb stairs, to walk more quickly or slowly, to stand up or sit down. However, care must be taken to ensure that the method can be conducted independently of the actual execution of the different states of movement by the person. The measurement data detected following a demand via the communication device is allocated to the second state of movement, regardless of whether the second state of movement has actually been assumed and correctly executed or not. As previously described, the method may be executed entirely by a computer that has access to the measurement data recorded by the at least one sensor.

It is preferable if the first state of movement is recognized from the first set of measurement and/or the second state of movement from the second set of measurement data. This may be achieved, for instance, by storing certain measurement data sequences, such as a knee angle sequence while walking, in an electronic memory. The recorded measurement data is then compared with a series of these different patterns of movement and data sequences until a match is found. The corresponding state of movement is recognized as a first state of movement or second state of movement and assigned accordingly to the measurement data.

The evaluation of the first and second set of measurement data is preferably followed by the detection of a corrective measure and preferably transmitted by the communication device. This corrective measure relates to the set-up of prosthesis and may consist of, for example, a displacement of the foot relative to the lower leg and/or the knee, or a different positioning of the individual components of the prosthesis relative to one another. However, the corrective measure may also or only consist of the replacement of a used component in the orthosis or prosthesis with another component, thereby replacing, for instance, a prosthetic foot in a leg prosthesis with another prosthetic foot. This ensures that the wearer of the prosthesis or orthosis not only receives and can use an optimally set-up prosthesis or orthosis, but also that this is a prosthesis or orthosis made up of as optimal a combination as possible of individual components.

The corrective measure is preferably transmitted to a component of the prosthesis or orthosis by the communication device. This may be achieved in a wired or wireless manner, for example via radio, WiFi or Bluetooth. The component of the orthosis or prosthesis is preferably configured to implement the corresponding signals and carry out the corrective measure contained within said signals as a response to the signals received. This may occur, for instance, by way of an increase or reduction in a bending resistance, a change in a position of a component or an adjustment of a damping. Safety-relevant changes and/or corrective measures are preferably to be approved by a person, for example an orthopedic technician.

The corrective measure is preferably as specific as possible, so that it can be easily implemented by an orthopedic technician. For instance, it may contain instructions regarding which screw or which adjustment mechanism has to be rotated in which direction or how it should be adjusted.

Additionally or alternatively to a corrective measure, a training recommendation may be emitted to communicate to the person the optimal manner in which to operate the prosthesis or orthosis.

It is preferable if technical properties and/or restrictions of the orthosis or prosthesis and/or ranges of movement and/or limitations of the person are taken into account during the evaluation of the first set of measurement data and the second set of measurement data. For instance, if a prosthetic knee joint in use cannot allow a stance phase flexion, this is taken into account during the evaluation of the measurement data. In this case, it clearly makes little sense to attempt a stance phase flexion by way of a corrective measure. If it still deemed beneficial and/or necessary, the corrective measure comprises the replacement of the knee joint instead. Restrictions of movement, such as restricted ranges of movement, of the person are preferably taken into account in order to achieve as optimal and individually determined a set-up as possible of the prosthesis or orthosis by way of the method. This data is preferably stored in a database and can be entered manually, for instance. Of course, the electronic data processing device has access to this database.

Such restrictions of the components used or of the patient are preferably tested prior to conducting the method and stored in a database to which the electronic data processing device has access. Alternatively, they can also be imported and stored via data transmission, for example by a storage medium or online.

It is beneficial if a bio-mechanical model of the person with the orthosis or prosthesis is stored and parameterized using the first set of measurement data and/or the second set of measurement data, said model then being used to calculate kinetic data from kinematic data.

The first set and/or second set of measurement data preferably originate at least also from sensors, which are fixed to an untreated limb of the person. Preferably, one part is fixed to the healthy, i.e. untreated, body part of the person, whereas another part is fixed to treated body parts, i.e. to the prosthesis itself. A symmetry of the gait pattern is preferably detected from the first set of measurement data and the second set of measurement data. Within the scope of present invention, a sensor that is arranged on the prosthesis is also considered as being fixed to a body part. Here, a body part should be understood to mean both a natural body part of a person and a component of an orthosis or prosthesis worn by the person.

Suitable sensors are, for example, an angle sensor, inertial sensor, pressure sensor, force and/or momentum sensor, video or image sensor. The combination of data from different sensors and/or different states of movement allows new information to be obtained, which would not be possible without this combination. The data from different sources is preferably merged.

This renders it possible to record and compare measurement data of both the treated and the untreated limb. This is particularly advantageous for observations of symmetry and symmetry measurements. This relates, for example, to the measurement of the stride length or the double stride length, as well as the duration of the stride or the duration of the double stride. This method can also be used to detect the gait speed, the maximum knee angle during the stance phase or the maximum knee angle during the swing phase for both limbs and compared with one another for symmetries. Should any asymmetries be recognized, this may be an indication that corresponding corrective measures should be undertaken. Further variables that can be detected by the at least one sensor include, for instance, the type of circumduction carried out by the patient, the maximum ground clearance in the swing phase of a step, the minimum knee angle in the stance phase, as well as the proportion—on a percentage basis—of the stance phase and/or the swing phase in the gait cycle, wherein the duration in particular is observed. Of course, cadence, course length and other parameters, such as knee angle speeds, may also be detected. During the evaluation of the measurement data, original measurement data detected in such a manner, which originates directly from the sensors, can be used to detect additional derived values, especially time derivatives or relative angles. All of this data and measurement data preferably forms the basis of the evaluation so as to ensure as optimal a prosthesis set-up as possible.

Preferably, the first state of movement or the second state of movement is standing, a slow walk, a fast walk, a movement upstairs or downstairs, walking along an incline or a decline, standing or sitting, standing up or sitting down, or shifting from foot to foot on the spot (an alternating raising of the legs). Acceleration also constitutes such a state of movement. It is beneficial if the first state of movement and the second state of movement also or only differ in a ground condition. Here, the movement of the first state of movement, for example walking on a plane, is executed on a different surface to the movement of the second state of movement. For instance, the movements may be conducted on hard surfaces such as stone, concrete or wood, or on soft surfaces such as sand, a forest floor, grass or a carpeted surface.

During the evaluation of the first set of measurement data and the second set of measurement data, they are preferably compared with stored reference data, said reference data preferably being stored in an electronic memory. This may refer to data taken from other persons, such as other patients with the same illness or disability and/or the same orthosis or prosthesis.

For this type of method, a system is preferably used that features at least one sensor to be fixed to a body part of a person and an electronic data processing device, which is configured to conduct a method described here, wherein the system preferably has a communication device, particularly one which is designed as a display. It is also the case here that a sensor that is arranged on a prosthesis or a component of the prosthesis is also considered to be a sensor on a body part of the person.

The electronic data processing device is preferably configured to independently detect and conduct a suitable function test. Known tests include a “Timed up and go test (TUG)”, a “functional reach test (FRT)”, a “2 minutes walk test (2MWT)” or a “four square step test (FSST)”.

Measurement data that has been recorded during walking, which then forms the first state of movement, and while shifting from foot to foot, which forms the second state of movement, can be used to determine the parellelism of the feet (position of the feet relative to one another) as well as the stride length symmetry and the symmetry of the duration of the stance phase. If all these parameters are known, which cannot all be obtained from the data from just one state of movement, it is possible to assess the flexion contracture, for example.

To be able to assess the swing phase extension and/or the swing phase flexion, measurement data is preferably recorded during slow walking (first state of movement) and during acceleration (second state of movement).

If, during the first state of movement, it is established that the person is attempting to compensate for malpositions with other movements, the second state of movement may also consist of a “relaxed” execution of the same movement, for instance walking.

In the following, examples of embodiments of the present invention will be explained in more detail by way of the attached drawings: They show:

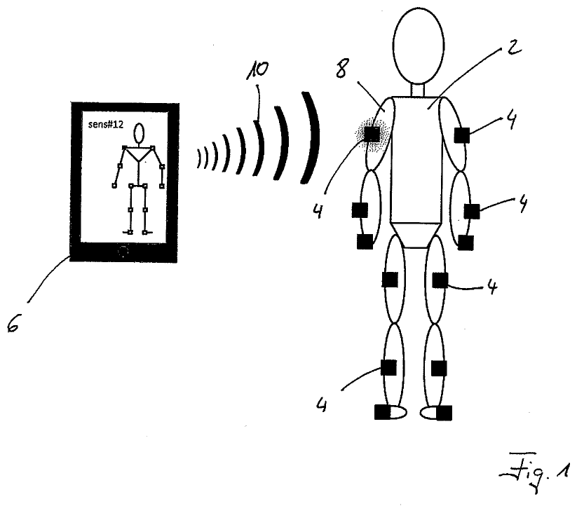

FIG. 1—a system with several sensors that are able to capture information on movements, positions and/or orientations in order to execute a method according to a first example of an embodiment of the present invention,

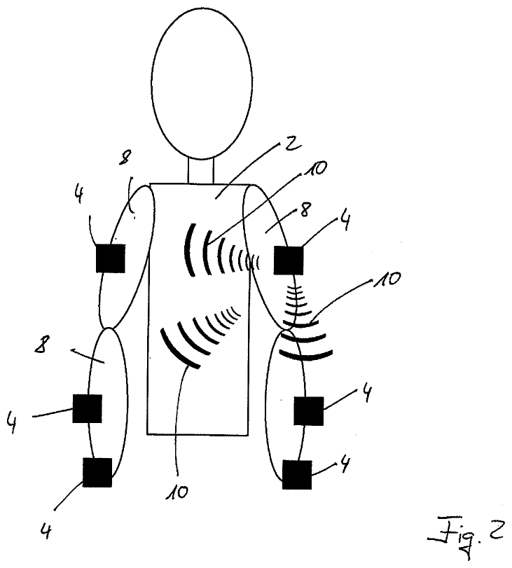

FIG. 2—a schematic view of a different system,

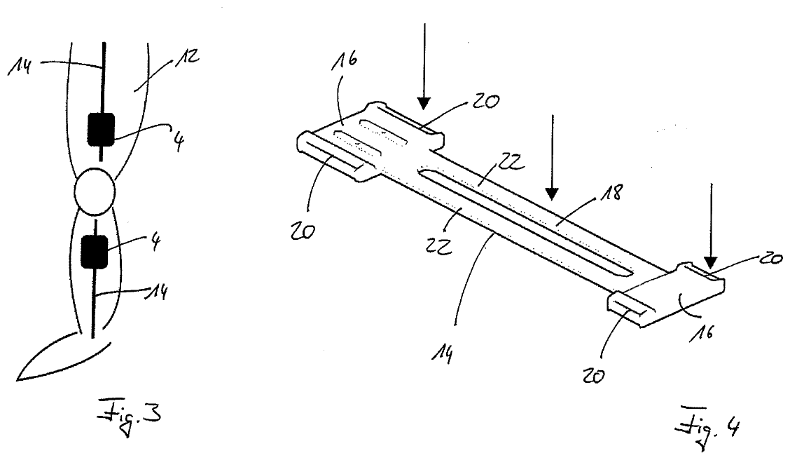

FIG. 3 the schematic depiction of a leg with two brackets according to an example of an embodiment of the present invention,

FIG. 4—the depiction of a bracket according to an example of an embodiment of the present invention,

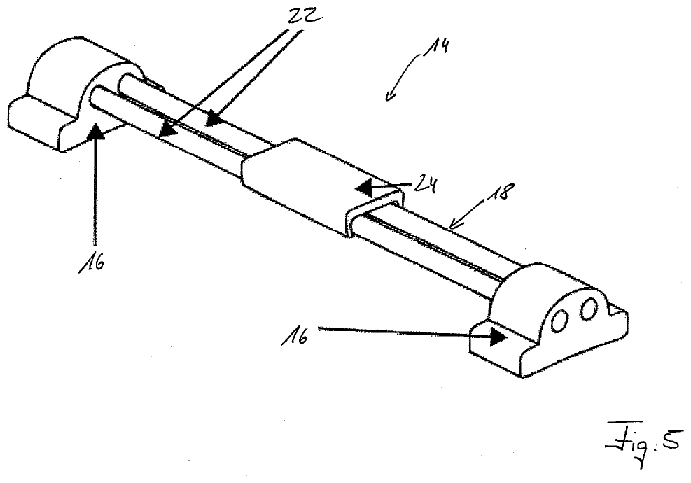

FIG. 5—the depiction of a bracket,

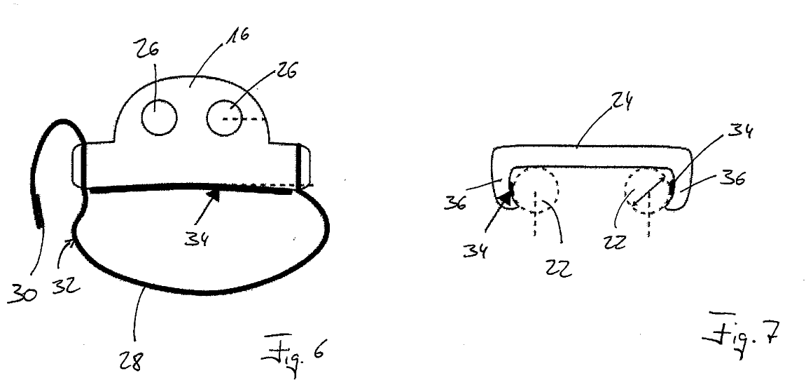

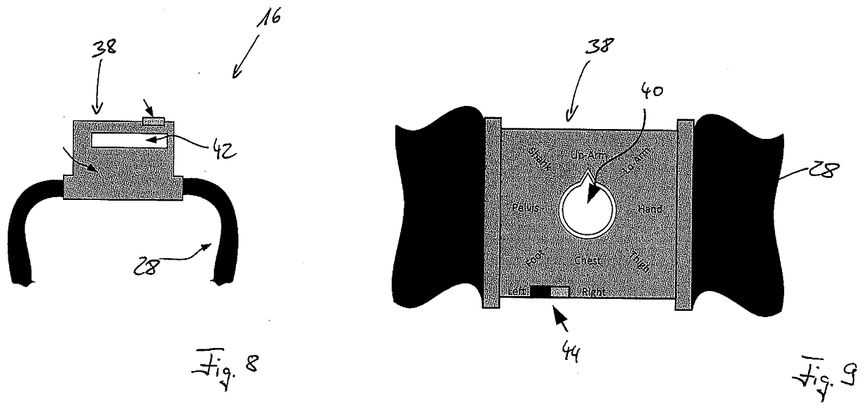

FIGS. 6 and 7—schematic details of the bracket from FIG. 5,

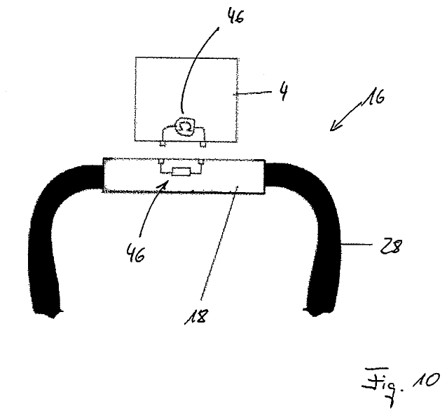

FIGS. 8 and 9—schematic depictions of fixing elements,

FIG. 10—the schematic depiction of a further bracket and

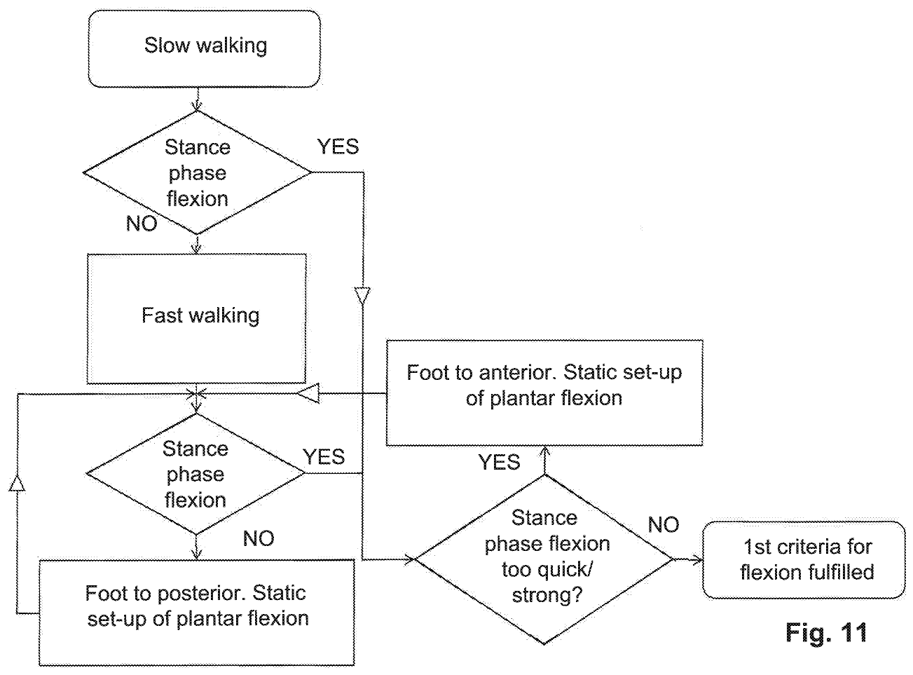

FIG. 11 the schematic view of a method.

FIG. 1 shows a person 2 to whose body a multitude of sensors 4 are fixed. The sensors 4 detect measured values, such as absolute angles, relative angles, speeds or accelerations and are preferably able to wirelessly transmit said values to an electronic data processing device 6. To ensure that the measured values determined by the sensors 4 are correctly evaluated in the electronic data processing device 6, the sensor must first of all be allocated to a body part 8. For the sake of clarity, only one body part 8 is illustrated, namely the upper arm of the person 2 shown on the left of FIG. 1.

To be able to achieve an allocation of the sensors 4 to the various body parts 8, the electronic data processing device 6—in the example of an embodiment shown—sends a signal 10, by means of which the sensor 4 arranged on the body part 8 is stimulated to emit a sensor signal or response signal.

FIG. 2 shows another embodiment in which the person 2 only has sensors 4 in the arm area. According to the example of the embodiment of the present invention, the sensors on the upper and lower arms in particular can be arranged on the upper and lower arms by way of brackets, which are described in more detail later. The sensor 4 depicted on the upper right of the upper arm in FIG. 2 emits signals 10 that can be detected by the other sensors 4. By determining run-times, distances between the sensors 4 can be determined, thereby also enabling the determination of the arrangements of the individual sensors 4 on the body parts 8 through the comparison of said distances with pre-defined, calculated or measured patterns.

FIG. 3 shows a schematic depiction of a leg 12 on which two sensors 4 are arranged. This is achieved via brackets 14 which may be configured according to the invention and are only schematically depicted in FIG. 3.

FIG. 4 shows such a bracket 14. It features two fixing elements 16 that are separated from one another by a spacer element 18. The two fixing elements 16 features slits 20, through which, for example, a fixing belt can be guided, so that the bracket 14 can be fixed to the respective body part 8.

In the example of an embodiment shown, the spacer element 18 has two individual elements 22 that are arranged next to one another. This ensures that the bending stiffness of the spacer 18 is considerably lower in a first direction than the bending stiffness in a second direction.

FIG. 5 shows a different configuration of the bracket 14. It also features two fixing elements 16, between which the spacer element 18 is situated, wherein said spacer element also features two individual elements 22. A sensor fixing element 24 is situated on these individual elements 22, wherein in the example of an embodiment shown said fixing element is designed such that it can be displaced along the individual elements 22 and thus along the spacer element 18.

FIG. 6 shows an enlarged depiction of one of the fixing elements 16 from another perspective. Two openings 26 can be seen in which the individual elements 22 are inserted. A belt 28 is situated on the fixing element 16, wherein said belt can be placed around a body part. In the example of an embodiment shown, a velcro element 30 is situated at the free end of the belt 28, wherein said velcro element can be attached to an outer side 32 of the belt 28, such that the belt 28 is closed and the fixing element 16 is arranged on the body part 8. In the example of an embodiment shown, an anti-slip coating 34 is provided on a side of the fixing element 16 facing the body part 8, by means of which a slipping of the fixing element 16 and therefore of the bracket 14 relative to the body part 8 should be prevented.

FIG. 7 depicts the sensor fixing element 24 and the individual elements 22, depicted by a dashed line only. The sensor fixing element 24 features two clamping arms 36 which at least partially enclose the individual elements 22 as depicted, thereby fixing the sensor fixing element 24 to the individual elements 22. In the example of an embodiment shown, an anti-slip coating 34 is provided between the individual elements 22 and the clamping arms 36 of the sensor fixing element 24, by means of which an inadvertent displacement and slipping of the sensor fixing element 24 relative to the spacer element 18 and its individual elements 22 is prevented.

FIGS. 8 and 9 show a fixing element 16 in the form of a belt 28. An adjustment device 38 with a control dial 40 is situated on said belt. The control dial can carry out adjustments depending on the body part 8 on which the sensor with this fixing element 16 is arranged. The corresponding identifier is shown on a small display 42; in the example of an embodiment shown, a slide controller 44 can also be used to adjust whether a sensor 4 arranged on a bracket 14, which is equipped with such a fixing element 16, is arranged on a right or left-hand side of the body.

By adjusting the control dial 40 and the slide controller 44, it is possible to adjust, for instance, a signal, which is emitted by the respective sensor 4 in response to a request signal 10, as depicted schematically in FIG. 1 for example. As a result, identical sensors can be used for, for example, different patients at different points and on different body parts 8, without having to adjust the sensors or the electronic data processing device.

FIG. 10 depicts a further embodiment of a fixing element 16, which also features a belt 28. The spacer element 18 is also shown in a side view, said spacer element being designed—in the example of an embodiment shown—in such a way that the sensor 4 can be arranged directly on the spacer element 18. Here, both the spacer element 18 and the sensor 4 have an electronic assembly 46, which can be brought into electrical contact by arranging the sensor 4 on the spacer element 18. This ensures that a signal, which is sent by the sensor 4 to an electronic data processing device 6, is modified in such a way that the electronic data processing device can determine from the signal information the type of sensor 4 and/or the position and orientation of the sensor 4, i.e. in particular it can determine the body part 8.

FIG. 11 depicts the schematic sequence of a method for configuring a prosthesis or orthosis or for determining malpositions in the set-up of a prosthesis or orthosis. The present method should check whether a prosthetic foot is correctly positioned in the anterior-posterior direction or whether it must be displaced in the anterior or posterior direction. To this end, the first measurement data is recorded in the first step in the method, characterized by “slow walking”. This preferably refers to the knee angle, i.e. the angle between the upper leg and the lower leg. This may be achieved, for instance, via two inertial angle sensors that measure the angle of the upper leg relative to the vertical and the angle of the lower leg relative to the vertical. Here, the vertical is the direction along the force of gravity. The thus recorded first set of measurement data is subsequently evaluated to determine whether a stance phase flexion is present. If this is not the case, the second set of measurement data is recorded in a further step of the method, characterized by “fast walking”. The fast walking constitutes the second state of movement. Here, slow walking and fast walking are not limited to certain ranges of walking speed. In each case, the designation only serves to indicate that the first state of movement of “slow walking” comprises a lower advancing speed than the second state of movement of “fast walking”. The walking itself occurs on a plane that is preferably perpendicular to the vertical, i.e. along the horizontal. This plane is preferably not tilted.

The thus recorded second set of measurement data are also examined to determine whether a stance phase flexion is present. A stance phase flexion refers to a bending of the knee during the first half of the stance phase in a gait cycle. Here, the heel strike is followed by a brief decrease in the knee angle, i.e. a flexion of the knee. If this does not occur in either state of movement, i.e. in neither set of recorded measurement data, it is recommended to displace the prosthetic foot in the posterior direction. In the case of a static set-up, the plantar flexion should then be adjusted. However, if a stance phase flexion is detected from the first set of measurement data and/or the second set of measurement data, it is necessary to check whether this flexion occurs within a reasonable speed range and exhibits sufficient strength. Too fast or too strong a stance phase flexion can be attributed to malpositions in the prosthetic set-up that must be corrected. To achieve this, it is recommended to displace the foot in the anterior direction and subsequently to preferably statically adjust the plantar flexion. This method shall be executed until the bending criteria has been fulfilled and the anterior-posterior alignment of the prosthetic foot has been determined to a sufficient degree. This method is preferably conducted on patients with transtibial amputations who consequently have a natural knee.

### REFERENCE LIST

2 person
4 sensor
6 electronic data processing device
8 body part
10 signal
12 leg
14 bracket
16 fixing element
18 spacer element
20 slit
22 individual element
24 sensor fixing element
26 opening
28 belt
30 velcro element
32 outer side
34 anti-slip coating
36 clamping arm
38 adjustment device
40 control dial
42 display
44 slide controller
46 electronic assembly

1. A method comprising:
fixing at least sensors to a body part of a person;
detecting, with the at least one sensor, measurement data about a distance between the two sensors;
sending signals to an electronic data processing device, the signals including the measurement data and an individual sensor identifier;
determining, with the electronic data processing device, the body part on which the sensors are arranged or is to be arranged and allocates the measurement data of the at least two sensors to the body part.

2. The method according to claim 1, wherein at least one detector is allocated to the electronic data processing device, the detector detecting at least one part of the signals, wherein the electronic data processing device determines the body part using the detected signals, and determines a direction or position from which the individual sensor identifier is sent, or from a time path of the measurement data.

3. The method according to claim 1, wherein the signals also contain information on the body part.

4. The method according to claim 3, wherein the sensors are fixed to the body part with a bracket, which has an effect on the signals sent by the sensors.

5. The method according to claim 4, wherein the sensors take the information on the body part from the bracket.

6. The method according to claim 4, wherein the bracket detects the body part to which it is fixed.

7. The method according to claim 3, wherein the sensor includes an adjustment device, the adjustment device adjusting the information on the body part.

8. The method according to claim 1, wherein the sensors only send the signals to the electronic data processing device if sensors have received a request signal.

9. The method according to claim 1, wherein the sensors are fixed to different body parts.

10. (canceled)

11. The method according to claim 9, wherein the sensors emit test signals that are detected by other sensors, and information on the signals detected in this way, especially the times of detection, are transmitted from the sensors to the electronic data processing device.

12. A method comprising:
fixing at least two sensors to a body part of a person;
detecting, with the at least one sensor, measurement data about a distance between the two sensors;
sending signals to an electronic data processing device, the signals containing the measurement data;
sending, with the electronic processing device, a control signal to the sensors and the sensors, in response to the control signal, emit a response signal that can be detected by a person.

13. A system comprising:
the sensors;
the electronic data processing device, the system being configured to conduct the method according to claim 1.

14. A method comprising:
fixing at least one sensor to a body part of a person;
sending, with the at least one sensor, signals to an electronic data processing device, the signals including measurement data and an individual sensor identifier;
determining, with the electronic data processing device, the body part on which the at least one sensor is arranged or is to be arranged and allocates the measurement data of the at least one sensor to the body part;
emitting, with the at least one sensor, test signals that are detected by other sensors, and information on the test signals detected in this way, especially during times of detection, are transmitted from the other sensors to the electronic data processing device.

15. The method according to claim 14, wherein a detector is allocated to the electronic data processing device, the detector detecting at least one part of the signals, wherein the electronic data processing device determines the body part using the detected signals, and determines a direction or position from which the individual sensor identifier is sent, or from a time path of the measurement data.

16. The method according to claim 14, wherein the signals contain information on the body part.

17. The method according to claim 16, wherein the at least one sensor is fixed to the body part with a bracket, the bracket having an effect on the signals sent by the at least one sensor.

18. The method according to claim 17, wherein the at least one sensor receives the information on the body part from the bracket.

19. The method according to claim 17, wherein the bracket is used to detect the body part to which it is fixed.

20. The method according to claim 16, wherein the at least one sensor includes an adjustment device, the adjustment device adjusting the information on the body part.

21. The method according to claim 14, wherein the at least one sensor only sends the signals to the electronic data processing device if the at least one sensor has received a request signal.
